(19) United States
(12) Patent Application Publication
(10) Pub. No.: US 2021/0213175 A1
(43) Pub. Date: Jul. 15, 2021

(54) URINARY CATHETERS INCLUDING MECHANICAL PROPERTIES THAT ARE STABLE OVER A RANGE OF TEMPERATURES

(71) Applicant: Hollister incorporated, Libertyville, IL (US)

(72) Inventors: Brent H. Sellers, Libertyville, IL (US); Horacio Montes de Oca Balderas, Ballina (IE)

(21) Appl. No.: 16/769,175

(22) PCT Filed: Dec. 4, 2018
(86) PCT No.: PCT/US2018/063857

§ 371 Date: Jun. 2, 2020

Related U.S. Application Data
(60) Provisional application No. 62/594,896, filed on Dec. 5, 2017.

(51) Int. Cl. A61L 29/14; A61L 29/04; C08L 53/00; C08L 23/22

(52) U.S. Cl. CPC A61L 29/14; A61L 29/041; A61M 25/0017; C08L 23/22; C08L 53/00

(57) ABSTRACT

A urinary catheter and materials for making a urinary catheter having mechanical properties, such as stiffness and flexibility, which are stable over a range of temperatures.

### CROSS REFERENCE TO RELATED APPLICATION

This application claims the benefit and priority of U.S. Patent Application Ser. No. 62/594,896, filed Dec. 5, 2017, which is hereby incorporated by reference in its entirety.

### DESCRIPTION

### Technical Field

The present disclosure generally relates to urinary catheters that have a catheter tube that maintains the mechanical properties/features over a range temperatures and materials for forming the same.

### Background

Catheters are used to treat many different types of medical conditions and typically include an elongated catheter tube that is inserted into and through a passageway or lumen of the body. Catheters, and in particular intermittent catheters, are commonly used by those who suffer from various abnormalities of the urinary system, such as urinary incontinence. With the advent of intermittent catheters, individuals with urinary system abnormalities can self-catheterize (i.e., self-insert and self-remove intermittent catheters) several times a day.

Intermittent urinary catheters may be used in locations and climates that have significant temperature changes. For example, locations that have hot summers and cold winters. Also, catheter users may travel between hot and cold climates. Given the intimate nature and sensitivity of self-catheterization, some urinary catheter users may desire to have a consistent self-catheterization experience. One common issue that catheter users may face with self-catheterization is that in hotter temperatures or climates, the catheter tubes may become very soft which can change the user's experience and possibly hinder the user's ability to insert/advance the catheter. It is not uncommon for some catheter users to place catheters in a refrigerator prior to use to cool the catheter and increase the catheter's stiffness. In colder temperatures/climates the catheter tubes may become stiffer which can also change the user's experience and hinder the ability to insert/advance the catheter, especially in male users wherein the catheter must traverse the curved pathway of the urethra. It also is not uncommon for some catheter users to warm the catheter prior to use to increase the catheter's flexibility.

Therefore, there is a need for catheters that have catheter tubes in which the mechanical properties of the tube remain relatively stable over various temperatures and/or over a temperature range.

### SUMMARY

There are several aspects of the present subject matter which may be embodied separately or together in the devices and systems described and claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto.

In one aspect, a urinary catheter includes a catheter tube made from a polymeric material having a tan δ peak at 0° C. or less and a tensile storage modulus greater than about 10 MPa in the temperature range of 0° C. and 40° C. measured at 1 Hz oscillation frequency.

In another aspect, a urinary catheter including a polymeric urinary catheter tube which has a storage modulus that increases less than 500% when cooled from 40° C. to 0° C. as measured at 1 Hz oscillation.

### BRIEF DESCRIPTION OF THE FIGURES

FIG. 1 is a graph showing the change in storage modulus over temperature for materials of the Example.

FIG. 2 is a graph showing the tan δ over temperature for materials of the Example.

### DETAILED DESCRIPTION OF THE ILLUSTRATED EMBODIMENTS

The embodiments disclosed herein are for the purpose of providing a description of the present subject matter, and it is understood that the subject matter may be embodied in various other forms and combinations not shown in detail. Therefore, specific embodiments and features disclosed herein are not to be interpreted as limiting the subject matter as defined in the accompanying claims.

The present application discloses urinary catheter tubes wherein the mechanical properties of the tubes, such as the viscoelastic properties, are relatively stable over a range of temperatures. The present application also discloses polymeric materials having such stable mechanical properties. The stiffness of the polymeric materials and the catheter tubes made therefrom may be relatively stable at various different temperatures or over specific ranges temperatures. That is, the catheter tubes and polymeric materials may have relatively small changes in stiffness and flexibility at different temperatures. This may be beneficial to intermittent catheter users that use catheters in both warm and cold climates. This is because, the stiffness/flexibility of the catheter tube remaining relatively the same regardless of the temperature in which it is used can aid in creating a consistent user experience. Depending on where a catheter user resides or where the user travels to, the user may encounter temperatures anywhere between 0° C. and 40° C. The catheter tubes disclosed herein may or may not have a lubricious hydrophilic coating thereon any/or may be used with a gel-lubricant applied to the catheter prior to catheterization.

One feature of the mechanical stability of the material from which a catheter tube is made may be the difference in the change of the material's storage modulus over a temperature range. Another feature of the mechanical stability of the material from which the catheter tube is made may be the temperature of the tan delta (tan δ) peak of the material. Tan δ is defined as the ratio between the loss modulus and the storage modulus. The methods for measuring storage modulus and the temperature of the tan δ peak may, for example, employ a Dynamic Mechanical Analyzer. A TA Instruments DMA Q800, available from TA Instruments, Inc., of New Castle, Del.; equipped with a film clamp and Thermal Advantage software for data acquisition may be used for such data analysis. Many other types of DMA devices exist, and the use of dynamic mechanical analysis is well known to those skilled in the art of polymer and copolymer characterization. In one method of dynamic mechanical analysis the parameters may include oscillation frequency of 1 Hz, oscillation amplitude of 25.0, static force of 0.0100 N, forcetrack at 125.0% and minimum oscillation force at 0.02000 N. Furthermore, when measuring the mechanical properties of the materials that form a medical tube, such as a urinary catheter tube, the wall of the catheter tube may be cut into a rectangular sample having a length of 33 mm and a width of 6.5 mm. The mechanical properties may then be analyzed using the instruments described above. In one instance the rectangular sample of the material of the medical tube may have a thickness of 0.71 mm.

In one embodiment of a urinary catheter, the catheter may include a catheter tube made from a polymeric material having a tan δ peak at 0° C. or less. In another embodiment, the polymeric material may have a tan δ peak at −10° C. or less, or have a tan δ peak between about −15° C. and 0° C., or a tan δ peak of between about −15° C. and −10° C., or a tan δ peak of between about −20° C. and −15° C.

In addition to the above-mentioned tan δ peaks, the polymeric material from which the catheter is made may also have a storage modulus greater than about 10 MPa in the temperature range of 0° C. and 40° C. In one embodiment, the polymeric material may have a storage modulus of greater than about 20 MPa in the temperature range of 0° C. and 40° C. or between about 20 MPa and about 50 MPa in the temperature range of 0° C. and 40° C. In another embodiment, the polymeric material may have a storage modulus of greater than about 40 MPa in the temperature range of 0° C. and 40° C. or between about 40 MPa and about 200 MPa in the temperature range of 0° C. and 40° C.

As mentioned above, another mechanical feature of the polymer material, which may be in addition to the above features or independent of the above features, is that the increase of the storage modulus may be less than or equal to 500% when the material is cooled from 40° C. to 0° C. In another embodiment, the storage modulus increases less than 300% when cooled from 40° C. to 0° C. In another embodiment the storage modulus increases less than 200% when cooled from 40° C. to 0° C. In yet another embodiment, the ratio of the storage modulus at 0° C. to that of the storage modulus at 40° C. is between about 5:1 and about 2:1. Percentage increase of the storage modulus shall be understood in this context as follows: storage modulus at low temperature (e.g. 0° C.) divided by storage modulus at high temperature (e.g. 40° C.) multiplied by 100.

The polymeric materials from which at least a portion of the urinary catheter tubes are made may include, for example, a thermoplastic elastomer (TPE) or TPE blended with other polymers. The blending may be by physical blending, compounding or any other suitable bending method. Such blends may then be extruded or molded by any suitable methods to form a catheter tube. For instance, the TPE may be compounded with a polyolefin such as polypropylene, polyethylene, polyamide block copolymer, polyurethane, polyester, EVA, ethylene based plastomers and the like. These blends in turn can also be compounded with solid fillers and/or liquid additives to impart other functionalities to the blend. When the polyolefin is polypropylene, it may be, for example, syndiotactic polypropylene or random copolymer polypropylene. In one embodiment, the blends may have a melting point of about 140° C.

The TPE may be an elastomer, including but not limited to, block copolymers. The block copolymer may be, for example, a styrenic block copolymer TPE. Such stryenic block copolymer TPEs may include styrene-butadiene-styrene (SBS) or styrene-ethylene-butylene-styrene (SEBS). Such SEBS and SBS TPE materials are commonly, but not exclusively, sold under the tradename Kraton®. In one embodiment, the SEBS may have a styrene content of greater than 20%, or between about 20% and about 70%, or between about 40% and about 60%. The TPE also may be a poly(1-butene) based plastomer, such as Purell KT MR 07, and/or copolymers thereof.

In one embodiment, the TPE and polyolefin blend may include TPE in the amount of between 5 wt % and 95 wt % of the polymeric material and the polyolefin may be between about 5 wt % and about 95 wt % of the polymeric material. In another embodiment, the TPE may be between about 40 wt % and about 70 wt % and the polyolefin may be between about 30 wt % and about 60 wt % or the TPE may be between about 45 wt % and about 65 wt % and the polyolefin may be between about 35 wt % and about 55 wt %. In yet another embodiment, the TPE may be about 50 wt % and the polyolefin may be about 50 wt %. For example, in one embodiment, the material of the catheter tube may be between about 40 wt % and about 60 wt % poly(1-butene) or copolymers thereof, and between about 40 wt % and about 60 wt % polypropylene.

### EXAMPLE

The storage modulus and the tan δ the materials of various catheter tubes were measured using a TA Instruments Q-800 DMA and Thermal Advantage V7.5 Build 127 software. The parameters of the testing instrument were oscillation frequency of 1 Hz, oscillation amplitude of 25.0, static force of 0.0100 N, forcetrack at 125.0% and minimum oscillation force at 0.02000 N.

To measure the materials of the catheter tubes, a rectangular sample was cut from each of the tubes. The rectangular sample had the approximate dimensions of: length ˜33 mm, width ˜6.5 mm and thickness ˜0.71 mm. The sample was then placed in a tension clamp and the measurements were taken.

Samples of materials were cut from five catheter tubes. Four of the catheter tubes (Samples 1-4) were made from materials in accordance with the present disclosure and one catheter tube (Sample 5) was a commercially available catheter for comparative purposes.

TABLE 1

| Sample | Material of Catheter |
|---|---|
| 1 | Wittenburg PR12662 E Shore 80A: SEBS W (80A) |
| | Supplied by Wittenburg B. V. |
| 2 | Wittenburg PR12662 D2 Shore 90A: SEBS W (90A) |
| | Supplied by Wittenburg B. V. |
| 3 | 60 wt % Purell KT MR 07/40 wt % Purell RP270G: |
| | PB-1 (80A) |
| | Supplied by LyondellBasell |
| 4 | 45% Purell KT MR 07/55% Purell RP270G: PB-1 |
| | (90A) |
| | Supplied by LyondellBasell |
| 5 | Lofric ® Ch14 TPE catheter: Lofic ® |
| | Supplied by Wellspect/Dentsply IH AB |

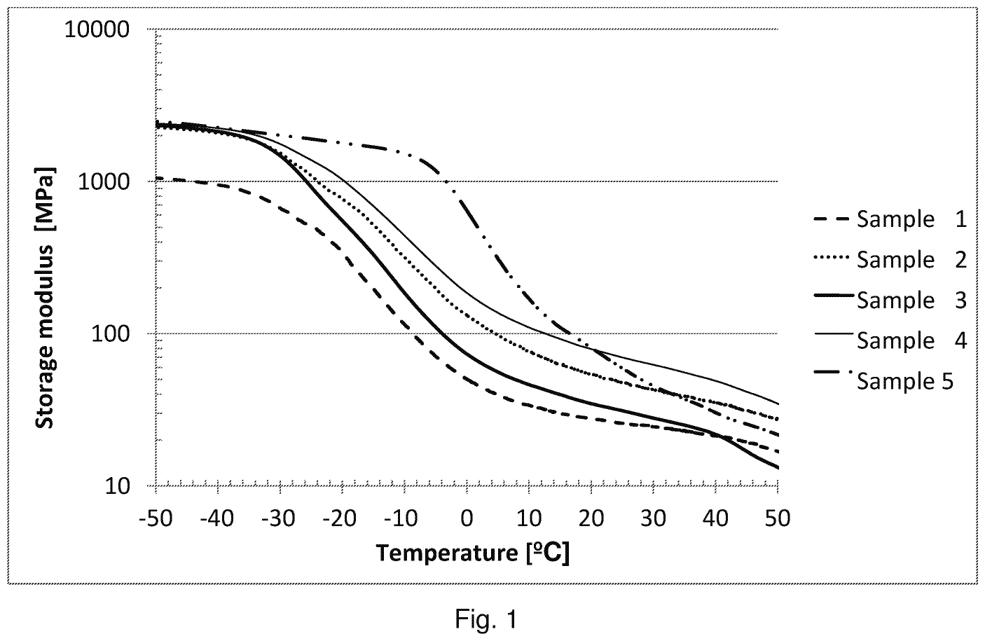

Table 2 below shows the storage modulus of each of the materials at 0° C. and 40° C. and the percent change of the storage modulus between these two temperatures. FIG. 1 includes a graph of the storage modulus over temperature.

TABLE 2

| | E′ | E′ | |
|---|---|---|---|
| | [Mpa] | [Mpa] | |
| | 0 C. | 40 C. | % |
| Sample 1 | 50 | 21 | 138 |
| Sample 2 | 132 | 35 | 277 |
| Sample 3 | 73 | 21 | 248 |
| Sample 4 | 185 | 49 | 278 |
| Sample 5 | 636 | 30 | 2020 |

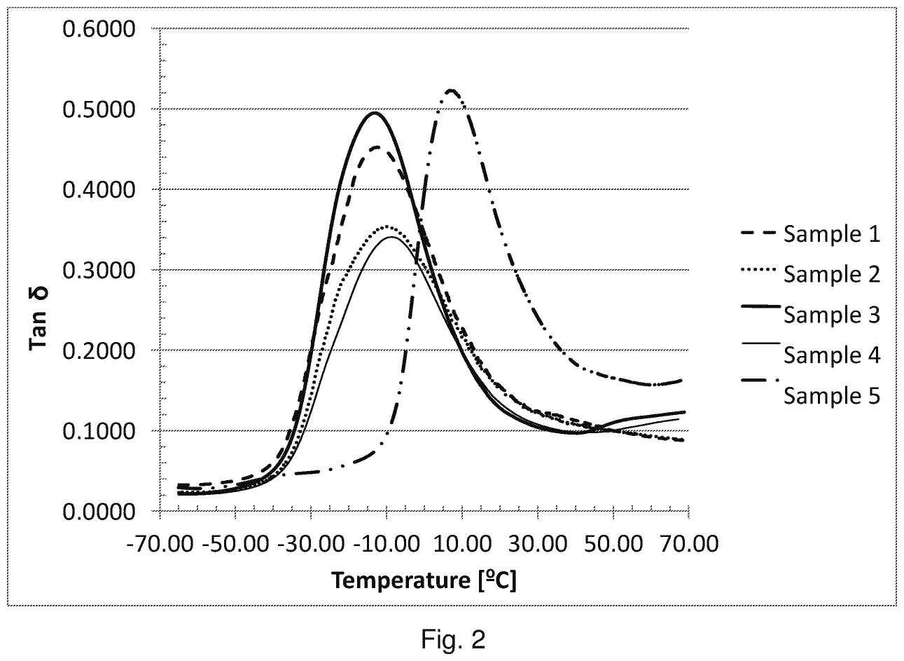

FIG. 2 includes a graph of the tan δ for each of the materials. As shown in the graph, each of Samples 1-4 has a tan δ peak at a temperature less than 0° C. and Sample 5 has a tan δ peak at a temperature above 0° C.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

1. A urinary catheter, comprising:
a catheter tube made from a polymeric material having a tan δ peak at 0° C. or less and a storage modulus greater than about 10 MPa in the temperature range of 0° C. and 40° C. measured at 1 Hz oscillation frequency.

2. The urinary catheter of claim 1 wherein a ratio of the storage modulus at 0° C. to the storage modulus at 40° C. is between about 5 to 1 and about 2 to 1.

3. The urinary catheter of claim 1 wherein the storage modulus increases less than 500% when the polymeric material of the catheter tube is cooled from 0° C. to 40° C.

4. The urinary catheter of claim 1 wherein the polymeric material comprises a thermoplastic elastomer.

5. The urinary catheter of claim 1 wherein the polymeric material comprises poly(1-butene).

6. The urinary catheter of claim 5 wherein the poly(1-butene) is in a copolymer.

7. The urinary catheter of claim 4 wherein the thermoplastic elastomer comprises a block copolymer.

8. The urinary catheter of claim 7 wherein the block copolymer comprises a styrenic block copolymer.

9. The urinary catheter of claim 7 wherein the block copolymer comprises styrene ethylene butylene styrene polymer or styrene butylene styrene.

10. The urinary catheter of claim 9 wherein styrene is an amount above about 40%.

11. The urinary catheter of claim 4 wherein the polymeric material further comprises a polyolefin.

12. The urinary catheter of claim 11 wherein the thermoplastic elastomer and polyolefin comprise a blend in which the thermoplastic elastomer is between 5 wt % and 95 wt % of the polymeric material and the polyolefin is between about 5 wt % and about 95 wt % of the polymeric material.

13. The urinary catheter of claim 11 wherein the polyolefin comprises polypropylene.

14. The urinary catheter of claim 13 wherein the polypropylene comprises syndiotactic polypropylene and/or random copolymer polypropylene.

15. The urinary catheter of claim 1 further including a lubricious hydrophilic coating disposed on an outer surface of the catheter tube.

16. A urinary catheter, comprising:
a urinary catheter tube that is made from a polymeric material which has a storage modulus that increases less than 500% when cooled from 40° C. to 0° C. as measured at 1 Hz oscillation.

17. The urinary catheter of claim 16 wherein the polymeric material comprises a thermoplastic elastomer.

18. The urinary catheter of claim 17 wherein the thermoplastic elastomer comprises poly(1-butene).

19. The urinary catheter of claim 18 wherein the poly(1-butene) is in a copolymer.

21.-29. (canceled)

30. The urinary catheter of claim 1 wherein the melting temperature of the polymeric material is about 140° C.
